(19) United States
(12) Patent Application Publication
(10) Pub. No.: US 2008/0171757 A1
(43) Pub. Date: Jul. 17, 2008

(54) Methods to identify inhibitors of cell sickling

(75) Inventors: William A. Eaton, North Bethesda, MD (US); H. J. Hofrichter, Adanstown, MD (US); Jeffrey F. Smith, Berkeley Heights, NJ (US)

Correspondence Address: EDWARDS ANGELL PALMER & DODGE LLP;(CLIENT REFERENCE NO. 47992), PO BOX 55874, BOSTON, MA, 02205, US

(73) Assignee: Government of the US, as represented by the Secretary, Department of Health and Human Serivces, Rockville, MD (US)

(21) Appl. No.: 11/652,843

(22) Filed: Jan. 11, 2007

(51) Int. Cl. G01N 33/48; A61K 31/341; A61K 31/522

(52) U.S. Cl. 514/263.37; 436/63; 514/461

(57) ABSTRACT

The invention relates to methods of screening for inhibitors of diseases or biological processes that involve peptide or protein polymerization. Included in the methods of the invention are methods to identify inhibitors of deoxyhemoglobin S polymerization. Also included in the invention are diagnostic and therapeutic methods.

### STATEMENT OF RIGHTS TO INVENTION MADE UNDER FEDERALLY SPONSORED RESEARCH

This work was supported by funds from the intramural research program of NIDDK, NIH. The government may have certain rights in the invention.

### FIELD OF THE INVENTION

The invention relates to methods of screening for inhibitors of diseases or biological processes that involve peptide or protein polymerization. The peptide or protein polymerization can be abnormal peptide or protein polymerization. Included in the methods of the invention are methods to identify inhibitors of Hemoglobin S polymerization. Also included in the invention are diagnostic and therapeutic methods.

### BACKGROUND OF THE INVENTION

Despite considerable knowledge regarding the etiology of sickle cell anemia, effective treatment has been elusive. Modalities for therapy have largely been directed at symptomatology. This disease is a major cause of illness in people whose ancestors come from areas such as sub-Saharan Africa, the Arabian Peninsula, the Indian subcontinent and the Mediterranean region. It is estimated that 1 in 600 black individuals suffer from this disorder and that 8% are heterozygous carriers of the trait. Moreover, an equal number of individuals suffer from sickle cell disease, including Sickle-Hemoglobin C disease (Sickle SC Disease), Sickle Beta=Plus Thalassemia or Sickle Beta-Thalassemia.

Despite the fact that the molecular basis of sickle cell disease was first understood by Pauling and colleagues in 1949 (2, 25), and numerous studies since have elucidated the mechanism of polymerization (13) and the structure of sickle hemoglobin fibers (26), very little progress has been in made in the discovery of new drugs to treat sickle cell disease. Previous efforts at discovering drugs for sickle cell disease (27-29) have been limited to investigating individual compounds, usually selected on the basis of anecdotal evidence, for their ability to retard the sickling process. At present, perhaps the drug used most frequently in the treatment of sickle cell disease is hydroxyurea, a commonly used chemotherapeutic agent (7). Treatment with hydroxyurea depends primarily on induction of the biosynthesis of intracellular hemoglobin F (HbF) (7), a hemoglobin known to be effective in inhibiting sickling both in vitro and in vivo. Clinical trials with hydroxyurea have demonstrated a reduction in frequency and severity of painful crises and in transfusion requirements (7,32). Despite the benefits of hydroxyurea therapy, there is concern regarding the consequences of long-term use of an anti-neoplastic agent, and treatment is far from optimal. U.S. Pat. No. 6,946,457, incorporated by reference herein in its entirety, is related to uses of antiviral agents such as acyclovir and valacyclovir to inhibit the aggregation of HbS and the sickling of erythrocytes taken from patients with sickle cell disease both in vitro and in vivo.

However, heretofore, the lack of a rapid, accurate, sensitive and scalable assay to measure sickling times has severely hindered the drug discovery process for sickle cell disease.

Thus, there remains a need in the art for methods of detecting peptide or protein polymerization, for example cell sickling, in a cell, in order to identify inhibitors and methods of treatment of diseases such as cell sickle disease.

### SUMMARY OF THE INVENTION

The invention provides, generally, methods for detecting cell sickling, peptide or protein polymerization. Included in the invention are methods to identify inhibitors of peptide or protein polymerization, methods to identify inhibitors of cell sickling, and methods to treat a subject having a disease or disorder of peptide or protein polymerization, for example cell sickling. Also included in the invention are compositions comprising an agent that inhibits peptide or protein polymerization, compositions comprising an agent that inhibits cell sickling. Kits according to the methods of the invention are also provided.

Accordingly, in one aspect, the invention provides a method for detecting peptide or protein polymerization, the method comprising inducing polymerization in a cell population, obtaining images of the cell population, and analyzing the images to determine the kinetics of sickling of the cells, thereby detecting peptide or protein polymerization in the cell population.

In another aspect, the invention provides a method for detecting cell sickling, the method comprising inducing polymerization in a cell population, obtaining images of the cell population, and analyzing the images to determine the kinetics of sickling of the cells, thereby detecting cell sickling in the cell population.

In another aspect, the invention provides a method for identifying an inhibitor of peptide or protein polymerization, the method comprising inducing polymerization in a cell population, contacting the cell population with a candidate agent, and determining the kinetics of cell sickling, thereby identifying an inhibitor of peptide or protein polymerization.

In still another aspect, the invention provides a method for identifying an inhibitor of cell sickling, the method comprising inducing polymerization in a cell population, contacting the cell population with a candidate agent, and determining the kinetics of cell sickling, thereby identifying an inhibitor of cell sickling.

In a further aspect, the invention provides a method for detecting peptide or protein polymerization, the method comprising inducing a process that effects cell sickling, obtaining images of the cell population, and analyzing the images to determine the kinetics of sickling of the cells, thereby detecting peptide or protein polymerization in the cell population.

In a further aspect, the invention provides a method for detecting cell sickling, the method comprising inducing a process that effects cell sickling, obtaining images of the cell population, and analyzing the images to determine the kinetics of sickling of the cells, thereby detecting cell sickling in the cell population.

In still a further aspect, the invention provides a method for identifying an inhibitor of peptide or protein polymerization, the method comprising, inducing a process that effects cell sickling, contacting the cell population with a candidate agent, and determining the kinetics of cell sickling, thereby identifying an inhibitor of peptide or protein polymerization.

In one aspect, the invention provides a method for identifying an inhibitor of cell sickling, the method comprising, inducing a process that effects cell sickling, contacting the cell population with a candidate agent, and determining the kinetics of cell sickling, thereby identifying an inhibitor of cell sickling.

In a related embodiment of the above-mentioned aspects, the chemical induction of polymerization is chemical induction with an agent that effects the production of deoxyhemoglobin. In one embodiment, the agent that effects the production of deoxyhemoglobin is sodium dithionite. In another related embodiment of the above-mentioned aspects, the light induction of polymerization is by aiming a light source at a cell population. In a particular embodiment, the light source is a laser beam.

In one embodiment of any of the above-mentioned aspects, the step of inducing polymerization in a cell population activates polymerization of deoxyhemoglobin-S, mutant Huntingtin protein or alpha-synuclein.

In one embodiment of any of the above-mentioned aspects, the step of inducing a process that effects cell sickling is selected from the method consisting of light induction, chemical induction, and slow deoxygenation.

In another aspect, the invention relates to a method for detecting peptide or protein polymerization, the method comprising aiming a light source at a cell population, obtaining images of the cell population, and analyzing the images to determine the kinetics of sickling of the cells, thereby detecting peptide or protein polymerization in the cell population.

In another aspect, the invention relates to a method for detecting cell sickling, the method comprising aiming a light source at a cell population, obtaining images of the cell population, and analyzing the images to determine the kinetics of sickling of the cells, thereby detecting cell sickling in the cell population.

In still another aspect, the invention relates to a method for identifying an inhibitor of peptide or protein polymerization, the method comprising aiming a light source at a cell population, contacting the cell population with a candidate agent, and determining the kinetics of cell sickling, thereby identifying an inhibitor of peptide or protein polymerization.

In a further aspect, the invention relates to a method for identifying an inhibitor of cell sickling, the method comprising aiming a light source at a cell population, contacting the cell population with a candidate agent, and determining the kinetics of cell sickling, thereby identifying an inhibitor of cell sickling.

In one embodiment of any of the above-mentioned aspects, the step of inducing polymerization in a cell population activates polymerization of deoxyhemoglobin-S, mutant Huntingtin protein or alpha-synuclein.

In one embodiment of any of the above-mentioned aspects, the light source is a laser beam. In another embodiment, the laser beam is a defocused laser beam. In another embodiment, the defocused laser beam is a continuous wave laser operating at wavelengths between 440 nm and 700 nm. In a related embodiment, the defocused laser beam is selected from the group consisting of: a gas laser, a semiconductor laser, a solid-state laser, a chemical laser, an excimer laser, a disk and fiber laser, or a dye laser. In a further related embodiment, the gas laser is selected from the group consisting of: an argon ion laser, a krypton laser, and a helium neon laser. In certain embodiments, the laser is a neodymium-doped yttrium aluminum garnet (Nd: YAG) laser. In other embodiments, the step of aiming the laser at a cell population activates polymerization of deoxyhemoglobin-S.

In another embodiment of any of the above-mentioned aspects, the kinetics of cell sickling is compared to the kinetics of cell sickling in a cell population that was not treated with agent.

In another particular embodiment of any of the above-mentioned aspects, the step of determining the kinetics of cell sickling further comprises the steps of i) obtaining images of the cell population and ii) analyzing the images to determine the kinetics of cell sickling. In one embodiment, the images of the cell population are obtained using a light source. In another embodiment, the analysis of the images is digital. In a further embodiment, the digital analysis comprises measuring the size and shape of cells in the population. In one embodiment, the size is measured by the radius of gyration of the cell. In another embodiment, the shape is measured by the skewness of the radial distribution of pixels in a cell.

In another aspect, the invention features a method for identifying an inhibitor of peptide or protein polymerization, the method comprising, inducing polymerization in a cell population, contacting the cell population with a candidate agent, and obtaining images of the cell population, and analyzing the images to determine the kinetics of cell sickling, wherein the analysis provides identification of an inhibitor of peptide or protein polymerization in the cell.

In another aspect, the invention features a method for identifying an inhibitor of cell sickling, the method comprising inducing polymerization in a cell population, contacting the cell population with a candidate agent, and obtaining images of the cell population, and analyzing the images to determine the kinetics of cell sickling, wherein the analysis provides identification of an inhibitor of cell sickling in the cell population.

In one embodiment of the above method, the step of inducing polymerization in a cell population is selected from the method consisting of: light induction, chemical induction, and slow deoxygenation. In a related embodiment, the chemical induction of polymerization is chemical induction with an agent such as sodium dithionite or any other chemical compound that can effect the transition of hemoglobin to the deoxygenated state. In another related embodiment, the light induction of polymerization is by aiming a laser at a cell population.

In another embodiment of the above method, the step of inducing polymerization in a cell population activates polymerization of a cellular component, such as deoxyhemoglobin-S.

In another aspect, the invention features a method for identifying an inhibitor of peptide or protein polymerization, the method comprising aiming a light source at a cell population, contacting the cell population with a candidate agent, obtaining images of the cell population, and analyzing the images to determine the kinetics of cell sickling, wherein the analysis provides identification of an inhibitor of peptide or protein polymerization in the cell.

In another aspect, the invention features a method for identifying an inhibitor of cell sickling, the method comprising aiming a light source at a cell population, contacting the cell population with a candidate agent, obtaining images of the cell population, and analyzing the images to determine the kinetics of cell sickling, wherein the analysis provides identification of an inhibitor of cell sickling in the cell population.

In one embodiment of any of the above-mentioned aspects, the light source is a laser beam. In another embodiment, the laser is a defocused laser beam. In a related embodiment, the defocused laser beam is a continuous wave laser operating at wavelengths between 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, and 740 nm. In preferred embodiments, the laser operates at wavelengths between 440 nm and 700 nm. In another embodiment, the defocused laser beam is selected from the group consisting of a gas laser, a semiconductor laser, a solid-state laser, a chemical laser, an excimer laser, a disk and fiber laser, or a dye laser. In a particular embodiment, the gas laser is selected from the group consisting of: an argon ion laser and a helium neon laser. In another embodiment, the step of aiming the laser at a cell population activates polymerization of deoxyhemoglobin-S, mutant Huntingtin protein or alpha-synuclein.

In one embodiment of any of the above-mentioned aspects, the kinetics of cell sickling is compared to the kinetics of cell sickling in a cell population that was not treated with agent. In another embodiment of any of the above-mentioned aspects, the step of determining the kinetics of cell sickling further comprises the steps of i) obtaining images of the cell population and ii) analyzing the images to determine the kinetics of cell sickling. In a particular embodiment, the images of the cell population are obtained using a light source. In another particular embodiment, the analysis of the images is digital. In a related embodiment, the digital analysis comprises measuring the size and shape of cells in the population. In another related embodiment, the size is measured by the radius of gyration of the cell. In another related embodiment, the shape is measured by the skewness of the radial distribution of pixels in a cell.

In another aspect, the invention features a method for identifying a subject having a disorder of peptide or protein polymerization, the method comprising inducing polymerization in a cell population, obtaining images of the cell population, and analyzing the images to determine the kinetics of cell sickling, thereby identifying a subject having a disorder of peptide or protein polymerization.

In another aspect, the invention features a method for identifying a subject having a disorder of peptide or protein polymerization, the method comprising aiming a light source at a cell population, obtaining images of the cell population, and analyzing the images to determine the kinetics of cell sickling, thereby identifying a subject having a disorder of peptide or protein polymerization.

In one embodiment, the light source is a laser beam. In another embodiment, the laser is a defocused laser beam. In a particular embodiment, the defocused laser beam is a continuous wave laser operating at wavelengths between 440 nm and 700 nm. In another particular embodiment, the defocused laser beam is selected from the group consisting of: a gas laser, a semiconductor laser, a solid-state laser, a chemical laser, an excimer laser, a disk and fiber laser, or a dye laser. In one embodiment, the gas laser is selected from the group consisting of: an argon ion laser and a helium neon laser. In another embodiment, the step of aiming the laser at a cell population activates polymerization of deoxyhemoglobin-S, mutant Huntingtin protein or alpha-synuclein.

In another aspect, the invention features a method for treating a subject having a disease or disorder of peptide or protein polymerization, the method comprising treating the subject with an effective amount of agent that inhibits peptide or protein polymerization, and thereby treating the subject having a disorder of peptide or protein polymerization.

In another aspect, the invention features a method for treating a subject at risk of developing a disease or disorder characterized by peptide or protein polymerization, the method comprising administering to the subject an effective amount of an agent that inhibits peptide or protein polymerization thereby treating the subject at risk of developing a disorder characterized by peptide or protein polymerization.

In one embodiment of any of the above aspects, the disorder of peptide or protein polymerization is a disease or disorder caused by or effecting changes in cell size or shape. In another embodiment, the disorder of peptide or protein polymerization is selected from the group consisting of sickle cell disease, Alzheimer's disease, Parkinson's disease, Huntington's disease, diseases associated with a prion protein, Hereditary Spherocytosis, and the class of diseases known as the hereditary amyloidoses. In still another embodiment, the disorder of peptide or protein polymerization is a disease associated with the presence of hemoglobin-S selected from the group consisting of: Sickle Cell Anemia, Sickle-Hemoglobin C disease, Sickle Beta-Plus-Thalassemia or Sickle Beta-Zero-Thalassemia.

In another embodiment, the invention features a method for analyzing the characteristics of a cell population for cell polymerization, the method comprising obtaining images of the cell population, and analyzing the images to determine the kinetics of sickling of the cells, thereby analyzing the characteristics of a cell population for cell polymerization.

In another embodiment, the invention features a method for analyzing the characteristics of a cell population for cell sickling, the method comprising obtaining images of the cell population, and analyzing the images to determine the kinetics of sickling of the cells, thereby analyzing the characteristics of a cell population for cell sickling.

In one aspect, the invention features a method of detecting a physical or chemical process, the method comprising inducing the physical or chemical process in a cell population, obtaining images of the cell population, and analyzing the images to determine the kinetics of the physical or chemical process, thereby detecting the physical or chemical process in the cell population.

In another aspect, the invention features a method of detecting an agent that inhibits or retards a physical or chemical process, the method comprising inducing the physical or chemical process in a cell population contacting the cell population with a candidate agent, obtaining images of the cell population, and analyzing the images to determine the kinetics of the physical or chemical process, thereby detecting an agent that inhibits or retards a physical or chemical process.

In another aspect, the invention features a method of detecting an agent that inhibits or retards a physical or chemical process, the method comprising inducing the physical or chemical process in a cell population, contacting the cell population with a candidate agent, obtaining images of the cell population, and analyzing the images to determine the kinetics of the physical or chemical process, thereby detecting an agent that inhibits or retards a physical or chemical process.

In one embodiment of any of the above-mentioned aspects, the images of the cell population are obtained using a light source. In a related embodiment, the analysis of the images is digital. In another related embodiment, the digital analysis of said images comprises measuring the size and shape of cells in the population. In another related embodiment, the size is measured by the radius of gyration of the cell. In still another related embodiment, the shape is measured by the skewness of the radial distribution of pixels in a cell. In a further embodiment, the analyzing of the images further comprises the steps of compiling time series data of the size or shape for each cell in the image. In another embodiment, the compiling time series data of the size or shape for each cell in the image further comprises the step of identifying the time at which the size or shape reaches a threshold value, or the time at which the rate of change of the size or shape with respect to time exhibits a maximum value.

In a further embodiment of any of the above-mentioned aspects, the method further comprises the step of obtaining a sample cell population. In another embodiment, the cell population is human red blood cells. In still another embodiment, the cell population is selected from the group consisting of: red blood cells, epithelial cells, cardiac cells, and neuronal cells.

In one aspect, the invention features a composition comprising an agent that inhibits peptide or protein polymerization, and a pharmaceutically acceptable carrier.

In another aspect, the invention features a composition comprising an agent that inhibits cell sickling, and a pharmaceutically acceptable carrier.

In one embodiment, the composition as described in the above-mentioned aspects further comprises one or more therapeutic agents.

In a further aspect, the invention features a kit comprising an agent that inhibits peptide or protein polymerization according to the methods as described herein, and instructions for use.

In a further aspect, the invention features a kit comprising an agent that inhibits cell sickling, and instructions for use.

### BRIEF DESCRIPTION OF THE DRAWINGS

The objects and features of the invention can be better understood with reference to the following detailed description and accompanying drawings.

FIG. 1 shows a schematic of the apparatus for the automated determination of sickling times of red blood cells. Bright field images of diluted red blood cells consisting of carboxy-hemoglobin in an anaerobic buffer were obtained in monochromatic light at 426 nm. An argon ion laser tuned to emit at 488 nm and aimed at a 45° angle relative to the sample was used to photodissociate the hemoglobin to the deoxygenated state in a large field of red blood cells, and thus serve as an optical trigger to initiate polymerization of Hemoglobin S (Hb S). Digital images were acquired on a CCD and analyzed subsequently using the algorithm described in the text to obtain sickling time distributions.

FIG. 2(a-e) shows determination of the sickling time for a single red blood cell. The radius of gyration and skewness were measured for each red blood cell in bright field images of a population of cells acquired during a laser photolysis experiment. Prior to the opening of the laser shutter 100 images were acquired in order to establish baseline values for the means and standard deviations of the distributions of the radius of gyration (R_{g}) and skewness (γ₁) for each cell. After the laser shutter was opened, carboxy-hemoglobin was photolyzed to deoxy-hemoglobin and polymerization was thus triggered. Bright field images with gray scale reversed and times relative to the opening of the laser shutter are shown in a-d. Changes in R_{g} (FIG. 2e, points labeled as dots) and γ₁ (FIG. 2e, points labeled as ‘x’) relative to the respective baseline mean values and normalized by the respective standard deviations were tracked for each cell as a function of time and are shown for the cell in the images shown in FIG. 2(a-d). For the curve corresponding to the composite of R_{g} and γ₁, Δ (FIG. 2e, points labeled as circles), the position of the maximal slope was located, and the sickling time (FIG. 2e, tₛ=11.3 s) was calculated by extrapolating the slope back to the time axis (FIG. 2e, Fit). The images in FIG. 2(a-d) show the appearance of the cell at the beginning of photolysis (a), at the sickling time (b) and one second thereafter (c), and at the end of the experiment (d). FIG. 3(a-f) shows the size and shape distributions of red blood cells before and after sickling. Bright field images of populations of red blood cells heterozygous for Hb S were acquired during a laser photolysis experiment, and cells were identified automatically using the procedure described in the text. The radius of gyration (R_{g}) and skewness (γ₁) were determined for each cell in each frame. Probability distributions (n=623) were calculated for R_{g} (FIG. 3a) and γ₁ (FIG. 3b) before (points labeled as ‘x’) and after (points labeled as dots) photolysis-induced sickling, and sickling is shown to induce a decrease in R_{g} (FIG. 3c) and an increase in γ₁ (FIG. 3d). Distributions before (right distribution in FIG. 3e and left distribution in FIG. 3f) and after (left distribution in FIG. 3e and right distribution in FIG. 3f) sickling for the particular cell as shown in FIG. 2 for R_{g} and γ₁ were also obtained.

FIG. 4(a-c) shows the determination of sickling time distributions for a population of red blood cells. Laser photolysis was used to trigger sickling in a population of red blood cells heterozygous for Hb S. The cells were diluted in a deoxygenated phosphate buffer that was determined to be 365 mOsm by the method of freezing point depression, subjected to carbon monoxide and delivered anaerobically to a flow cell. Bright field images of the cells at 37° C. were acquired during a single photolysis experiment (FIG. 4a and FIG. 4b, first and last images during photolysis), and the sickling time for each cell was determined as described in FIG. 2. The probability distribution of sickling times is shown (FIG. 4c), wherein the last bin (60 s, 6%) shows those cells determined to be unsickled at the end of the photolysis experiment.

FIG. 5(a-c) shows the reliability and repeatability of sickling time measurements. For each cell in the histogram in FIG. 5a, the distance of the cell's center of mass from the center of the image was calculated. To demonstrate that their was no significant correlation between the sickling time and position of the cell's center of mass from the center of the image the correlation coefficient for these two quantities was calculated (FIG. 5a, p=0.04, p=0.25). The laser power was also varied, and for each laser power a histogram (n>=50) of sickling times was obtained as in FIG. 4. The median sickling time of the histogram was plotted versus laser power, showing that full photodissociation of carbon monoxide was obtained with laser powers over ca. 10 mW. The laser power used in all other figures in this paper was 75 mW. The repeatability of the experiment was also examined. Two independent samples were prepared in an identical manner (365 mOsm buffer prepared as described in the methods, delivered anaerobically to a flow cell and maintained at 37° C.). Ten histograms were obtained for each sample, and each histogram was acquired on a different area of the sample. The cumulative probability distributions were plotted (FIG. 5c), and the distribution of median sickling times had a mean value of 3.1 s and standard deviation of 0.5 s.

FIG. 6(a-c) shows the sensitive detection of differences in sickling time distributions. Two dilutions of red blood cells heterozygous for Hb S were prepared in deoxygenated phosphate buffers, one with an osmolality of 365 mOsm and a second with an osmolality of 347 mOsm. For each sample, ten photolysis experiments were performed and each experiment was performed on a different spatial region. Sickling time distributions for each photolysis experiment were generated as in FIG. 4, and aggregated for each sample (FIG. 6a, 365 mOsm; FIG. 6b, 347 mOsm). The last bins in each probability distribution correspond to the number of unsickled cells at the end of the experiment. Comparison of the cumulative probability distributions (FIG. 6c) shows the exquisite sensitivity of intracelllular polymerization of Hb S on Hb S concentrations (FIG. 6c, solid curve, 365 mOsm; dotted curve, 347 mOsm), and the ability of the present method to detect readily the resulting changes in intracellular polymerization kinetics.

FIG. 7(a-f) shows the measurement of distortion magnitude, as measured by the normalized change in radius of gyration and skewness, and the measurement of the duration of growth phase during sickling. FIG. 7(a-b) shows that cells with the smallest distortion magnitude are associated with the shortest sickling times (e.g., sickling time less than 1 second). FIG. 7(c-d) shows that cells with the with the smallest distortion magnitude are associated with the shortest duration of the growth phase (e.g., transition time less than 1 second). FIG. 7e shows the correlation between the two measurements of distortion based upon changes in radius of gyration and skewness. FIG. 7f shows the correlation between the sickling time and the duration of the growth phase.

### DETAILED DESCRIPTION

The invention provides, generally, methods for detecting peptide or protein polymerization, for example cell sickling, in a cell. Included in the invention are methods to identify inhibitors of peptide or protein polymerization, and methods to treat a subject having a disease or disorder of peptide or protein polymerization, for example cell sickling. Also encompassed by any of the methods as described herein is treating a subject having a disease or disorder characterized by abnormal peptide or protein polymerization.

### Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the term “analyzing” means determining the kinetics of sickling of cells according to the methods described herein. “Analyzing” also means a method of measuring the size and shape of an identified object, for example, a cell. In a exemplary embodiment, the analysis encompasses measuring the radius of gyration of a cell and the skewness of the radial distribution of pixels in a cell. The “analysis” can be performed in a cell population, for example from a subject in order to identify a subject having a disorder of peptide or protein aggregation. The disorder can be one of abnormal peptide or protein aggregation. The “analysis” can also be performed in a cell population in order to identify, for example, agents that inhibit peptide or protein polymerization, or agents that inhibit cell sickling.

As used herein, the term “administer” or “administering” is defined to include an act of providing an agent, a compound or a pharmaceutical composition of the invention to a subject in need of treatment.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the term “effective amount” means an amount in sufficient quantities to accomplish the specific task, i.e., for instance either treat the subject, reduce or prevent sickling of cells and/or reduce or prevent polymerization of a peptide or protein. A person of ordinary skill in the art can perform simple titration experiments to determine what amount is required to treat the subject.

As used herein, “inhibits” means that the amount is reduced. Inhibition can mean a decrease or reduction. In preferred embodiments, an agent inhibits peptide or protein polymerization. In other preferred embodiments, an agent inhibits cell sickling. In other preferred embodiments, an agent inhibits Hb-S. Inhibition can be a percent inhibition, and can be, for example, a 10%, 20%, 30%, 40%, 45%, 50%, 55%, 60%, 65%, 70%, 75%, 80%, 85%, 90%, 95%, 96%, 97%, 98%, 99% or even 100% reduction or inhibition in peptide or protein polymerization, or cell sickling. Inhibition can be determined in fold-inhibition, for example, a 2-fold, 3-fold, 4-fold, 5-fold, reduction or inhibition in peptide or protein polymerization, or cell sickling.

As used herein, the term “polymerization” includes the process of forming a polymer from many monomeric units. In some embodiments of the invention, polymerization is of hemoglobin. A polymer may be formed by any chemical bonding interaction between or among molecules, i.e. covalent, ionic, or van der Waals. As used herein, “aggregation” and “polymerization” may be used interchangeably.

As used herein, the term “sample cell population” or “cell population” refers to a cell extract prepared from a whole organism or a subset of its tissues, cells or component parts, or a fraction or portion thereof. For example, a sample cell population according to some embodiments of the invention can be red blood cell samples. The sample cell population can be diluted in a buffer, for example a phosphate buffer, and prepared and stored according to art recognized methods.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “sickle cell disease” means that the subject has at least one cell that contains Hemoglobin S (Hb S). As used herein, a “sickle cell” includes an erythrocyte from a subject with sickle cell disease in which polymerization of Hb S has occurred. As used herein, the term “sickling” is meant to include the process whereby polymerization of Hb S occurs. As used herein, the term “sickled” is meant to refer to an erythrocyte from a subject with sickle cell disease in which polymerization of HbS has occurred.

As used herein, the by the term “subject” is meant a mammal, including, but not limited to, a human or non-human mammal, such as a bovine, equine, canine, ovine, or feline.

As used herein, “treating” a disease or disorder of peptide or protein polymerization, or abnormal peptide or protein polymerization, refers to either slowing, stopping or reversing the progression of peptide or protein polymerization. In preferred embodiments, “treating” refers to treating sickle cell disease. “Treating” as used herein can mean reversing the progression to the point of eliminating the presence of peptide or protein polymerization, or in preferred embodiments the presence of sickled cells. As used herein, “treating” also means the reduction in the amount of polymerization of hemoglobin or the amelioration of symptoms associated with sickle cell disease. Alternatively, “treating” is meant to refer to arresting or otherwise ameliorating symptoms of peptide or protein polymerization as, and in some examples abnormal peptide or protein polymerization, as defined herein.

### Sample Populations

The methods of the invention comprise obtaining a sample cell population. In certain embodiments of the invention, the cell population is selected from red blood cells, epithelial cells, cardiac cells, and neuronal cells. It may be, that, in further embodiments of the above methods, the cell is present in the subject, thus the methods of contacting the cell or cell population with an agent are effected by administering the agent to a subject. Subjects suitable for the invention include, but are not limited to, mammals, including, but not limited to, a human or non-human mammal, such as a bovine, equine, canine, ovine, or feline. The subjects may be patients, for example patients with a disease or disorder of protein or peptide polymerization. The subject may be a sickle cell disease patient.

### Methods of the Invention

Methods of Analysis

The polymerization of Hb S in red blood cells is characterized by the sickling time, the amount of time required for a detectable level of cellular distortion to occur. Several previous efforts have measured sickling times, including prior work that measured the fraction of sickled cells in a sample as a function of time (16, 17). However, this approach required data to be collected for a period of time of the order of hours, thus limiting throughput. Furthermore, this approach used buffers with dissolved oxygen, thus raising the possibility that observed inhibition of Hb S polymerization was due to the promotion of the oxy-hemoglobin state. An additional approach used a flow cell to introduce a solution containing sodium dithionite in order to trigger the deoxygenation of hemoglobin in red blood cells and used subjective criteria to determine sickling times for the cells in the captured video (18). However, this approach was not readily scalable given the need to reload the flow cell after each experiment. Another approach used laser photolysis of carboxyhemoglobin in an anaerobic buffer-to trigger sickling in individual cells (19), and the sickling time was detected by a microspectrophotometer. Although this method measured sickling times very accurately, it suffered from low throughput due its cell-by-cell approach. Therefore, none of the existing approaches could be used directly to test a large number of compounds for the ability to inhibit intracellular Hb S polymerization.

In one aspect, the invention teaches a method for analyzing the characteristics of a cell population for cell aggregation. The method comprises the steps of obtaining images of the cell population, and analyzing the images to determine the kinetics of sickling of the cells, thereby analyzing the characteristics of a cell population for cell aggregation. In another aspect, the invention teaches a method for analyzing the characteristics of a cell population for cell sickling, the method comprising obtaining images of the cell population, and analyzing the images to determine the kinetics of sickling of the cells, and thereby analyzing the characteristics of a cell population for cell sickling. In certain embodiments of these methods the images of the cell population can be obtained using a light source. Any source of light is suitable for use according to the methods of the invention, for example any monochromatic light source where the selected wavelength is in the range of about 410 nm to 440 nm. In preferred embodiments, the wavelength of the light source is 426 nm. In certain embodiments of these methods, the analysis of the images is digital. Digital images may be acquired on a charged coupled device (CCD) image sensor. Digital analysis, for example, can comprise measuring the size and shape of the cells in the population. Size may be measured, for example, by the radius of gyration of the cell. Shape may be measured, for example, by the skewness of the radial distribution of pixels in a cell. Accordingly, measurement of these two parameters (i.e. size and shape) are measured for each cell in the series of images and thus as a function of time. To analyze the images, further steps may be taken to compile time series data for each cell in the image. For each cell, fluctuations in the two parameters can be measured prior to polymerization, for example, deoxy-hemoglobin S polymerization. Baseline mean values and standard deviations are then calculated. Upon polymerization, e.g. deoxy-hemoglobin S polymerization, cells will begin to distort, and changes in the size and shape of cells, relative to the respective baseline mean values and standard deviations prior to polymerization can be observed. The point where the rate of change in these parameters was maximal can be identified, and the polymerization time, for example the sickling time, sickling time determined by extrapolating the slope from the measured relative change in the parameter values back to the point of intersection with the time axis. In analyzing the images, it may be necessary to have distributions of polymerization times. For example, given the extraordinary sensitivity of nucleation rates on Hb S concentration (21) and the distribution of hemoglobin concentration known to exist in red blood cells (22), it is necessary to have distributions of sickling times for a population of cells in order to be able to quantify properly intracellular Hb S polymerization.

In general, the methods of the invention are also applied as methods for detecting physical or chemical processes. For example, the methods as described herein may be used to detect a physical or chemical process. The method comprises inducing the physical or chemical process in a cell population, obtaining images of the cell population, and analyzing the images to determine the kinetics of the physical or chemical process, and thereby detecting the physical or chemical process in the cell population. The physical or chemical process can be a physical or chemical process that effects a change in cell size or shape.

Also encompassed by the invention are methods of detecting an agent that inhibits or retards a physical or chemical process. The method comprises inducing the physical or chemical process in a cell population, and contacting the cell population with a candidate agent, and obtaining images of the cell population, and then analyzing the images to determine the kinetics of the physical or chemical process, thereby detecting an agent that inhibits or retards a physical or chemical process. The physical or chemical process is a physical or chemical process that effects a change in cell size or shape.

Also possible are methods of detecting an agent that inhibits or retards a physical or chemical process. The method comprises inducing the physical or chemical process in a cell population, contacting the cell population with a candidate agent, obtaining images of the cell population, and analyzing the images to determine the kinetics of the physical or chemical process, and thereby detecting an agent that inhibits or retards a physical or chemical process. In this case, the analysis provides identification of an agent that inhibits or retards the physical or chemical process. In some embodiments, the physical or chemical process is a physical or chemical process that effects a change in cell size or shape.

Methods of Detecting Peptide or Protein Polymerization

The invention provides methods of detecting peptide or protein polymerization. Accordingly, the methods comprise inducing polymerization in a cell population, obtaining images of the cell population, and analyzing the images to determine the kinetics of sickling of the cells, and thereby detecting peptide or protein polymerization in the cell population.

The invention provides methods of detecting cell sickling. Accordingly, the methods comprise inducing polymerization in a cell population, obtaining images of the cell population, and analyzing the images to determine the kinetics of sickling of the cells, and thereby detecting cell sickling in the cell population.

The invention provides methods of detecting peptide or protein polymerization. Accordingly, the methods comprise aiming a laser at a cell population, obtaining images of the cell population, and analyzing the images to determine the kinetics of sickling of the cells, thereby detecting peptide or protein polymerization in the cell population.

The invention provides methods of detecting cell sickling. Accordingly, the methods comprise aiming a laser at a cell population, obtaining images of the cell population, and analyzing the images to determine the kinetics of sickling of the cells, thereby detecting cell sickling in the cell population.

### Screening Assays

The instant invention encompasses methods for identifying agents that inhibit peptide or protein polymerization. In certain embodiments, the methods if the invention are used to identify an inhibitor of cell sickling, for example a compound that inhibits intracellular Hemoglobin S (Hb S) polymerization.

Methods of Identifying Inhibitors

Included in the invention are methods for identifying inhibitors of peptide or protein polymerization. Accordingly, the methods comprise inducing polymerization in a cell population, contacting the cell population with a candidate agent, and then determining the kinetics of cell sickling, and wherein an inhibitor of peptide or protein polymerization in a cell population is identified. Polymerization can be induced by aiming a laser at a cell population.

Also included in the invention are methods for identifying an inhibitor of cell sickling. Accordingly, the methods comprise inducing polymerization in a cell population, contacting the cell population with a candidate agent, determining the kinetics of cell sickling, wherein an inhibitor of cell sickling is identified. Polymerization can be induced by aiming a laser at a cell population.

In any of the methods of the invention described herein, polymerization in a cell population can be induced by a number of means. For instance polymerization can be induced by light, chemical means, or slow deoxygenation.

Inducing polymerization in a cell population can activate polymerization of deoxyhemoglobin-S. Alternatively, proteins such as, but limited to, mutant Huntingtin protein or alpha-synuclein protein, or actin may be polymerized in a sample cell.

According to certain methods of the invention, polymerization can be induced by a chemical agent. Exemplary chemical agents that can be used to induce the polymerization of deoxyhemoglobin include, but are not limited to, sodium dithionite or any other chemical compound that can effect the transition of hemoglobin to the deoxygenated state. In certain embodiments, for example, Lysophosphatidic acid (LPA) has been shown to induce the polymerization of actin (33).

According to other certain methods of the invention, polymerization can be induced by slow deoxygenation. Slow deoxygenation can be defined as a process whereby a suspension of cells is placed in a sealed container and the dissolved oxygen in the buffer is consumed by the metabolic process of the cells.

According to certain methods of the invention, polymerization can be induced by a laser. In certain embodiments, the light induction of polymerization is carried out by aiming a laser at a cell population. For example, aiming the laser at a cell population activates polymerization of deoxyhemoglobin-S. The laser that is aimed at the cell population may be a defocused laser beam. By defocused laser beam is meant any laser beam that produces a corresponding larger illuminated field than a standard laser beam. Any laser beam is suitable for use according to the methods of the invention. In particular embodiments, the laser is a defocused laser, and in certain preferred examples the defocused laser beam is a continuous wave laser operating at wavelengths between 400 nm, 420 nm, 440 nm, 460 nm, 480 nm, 500 nm, 520 nm, 540 nm, 560 nm, 580 nm, 600 nm, 620 nm, 640 nm, 660 nm, 680 nm, 700 nm, 720 nm, and 740 nm. In another embodiment, the defocused laser beam is selected from the group consisting of a gas laser, a semiconductor laser, a solid-state laser, a chemical laser, an excimer laser, a disk and fiber laser, or a dye laser. In a particular embodiment, the gas laser is selected from the group consisting of: an argon ion laser and a helium neon laser. The laser can be a Nd:YAG (neodymium-doped yttrium aluminium garnet) laser. Nd:YAG crystal is the most widely used laser crystal on solid-state laser. Good fluorescence lifetime, thermal conductivity and robust nature make Nd:AYG crystals suitable for high power continuous wave, high intensity q-switched and single mode operation.

In order to identify inhibitors of peptide or protein polymerization, including inhibitors of cell sickling, the kinetics of peptide or protein polymerization, or the kinetics of cell sickling is compared in the population that was treated with agent to the kinetics of peptide or protein polymerization, or the kinetics of cell sickling in a cell population that was not treated with agent.

Any of the methods of the invention as described herein comprise the step of analyzing the images to determine the kinetics of cell sickling.

Also included in the invention are methods for identifying an inhibitor of peptide or protein polymerization. Accordingly, the methods comprise inducing polymerization in a cell population, contacting the cell population with a candidate agent, obtaining images of the cell population, and analyzing the images to determine the kinetics of cell sickling, wherein the analysis provides identification of an inhibitor of peptide or protein polymerization in the cell. Polymerization can be induced by aiming a laser at a cell population.

Also included in the invention are methods for identifying an inhibitor of cell sickling. Accordingly, the methods comprise inducing polymerization in a cell population, contacting the cell population with a candidate agent, obtaining images of the cell population, and analyzing the images to determine the kinetics of cell sickling, wherein the analysis provides identification of an inhibitor of cell sickling. Polymerization can be induced by aiming a laser at a cell population.

Methods of analysis to identify inhibitors of peptide or protein polymerization are described in detain in the section entitled “Methods of Analysis” above.

Candidate Agents

Potential candidate agents identified according to the methods of the invention include inhibitors of peptide or protein polymerization or inhibitors of cell sickling. Such inhibitors may include organic molecules, peptides, peptide mimetics, polypeptides, nucleic acid molecules (e.g., double-stranded RNAs, siRNAs, antisense polynucleotides), and antibodies that inhibit or otherwise reduce protein or peptide polymerization. Potential inhibitors might include small molecules, for example small organic molecules.

For example, small organic molecules may be identified that bind to HbS and interfere with the polymerization reaction. High resolution crystal structures of small molecule-hemoglobin complexes have been described in the art in an effort to design compounds that hinder HbS polymerization; however have not met with much success. For instance, Abraham et al. describe a number of compounds that have clear activity in inhibiting HbS polymerization (Abraham et al., 25 J. Med. Chem. 1015-17 (1982), and incorporated by reference herein). However, with the exception of a number of ethacrynic acid analogues, most of these compounds would have to be administered at rather high (ca. 5 mM) concentrations to achieve a therapeutically significant effect.

Small organic molecules have been used to bind to HbS noncovalently, thereby inhibiting the intermolecular interactions required for aggregation. Gorecki et al., 77 Proc. Nat. Acad. Sci., U.S.A. 181-85 (1980); Abraham et al., 27 J. Med. Chem. 967-78 (1984); Belie et al., 18 Biochemistry 4196-201 (1979); Elbaum et al., 71 Proc. Nat. Acad. Sci., U.S.A. 4718-22 (1974); Noguchi et al., 17 Biochemistry 5455-59 (1978); Ross et al., 77 Biochem. Biophys. Res. Comm. 1217-23 (1977). However, most compounds examined to date have shown limited potency in enhancing deoxy-HbS solubility.

Any number of methods are available for carrying out screening assays to identify new candidate compounds that inhibit peptide or protein polymerization.

High Throughput Screening

The development of new small molecule therapeutics typically begins with the identification of an active, or lead, compound that exhibits some of the properties required for safe and effective therapeutic intervention. Compounds with improved properties are subsequently derived through iterative cycles of analog preparation and testing. Lead compounds are often identified using high throughput screening (HTS), whereby large libraries containing 50,000 to 1,000,000 compounds are tested using relatively simple assays to measure inhibition of processes critical to the target indication. Typically this means using biochemical assays to measure the function of one or more macromolecular targets

In the instant invention, the combination of an optical trigger to initiate polymerization of Hb S and an analysis of digital images can be used to measure accurately the sickling times of large populations of human red blood cells in a manner that is readily scalable and amenable to screening for potential inhibitors of sickle cell disease.

Many approaches known in the art for measuring polymerization are not readily scalable given the need to reload the flow cell after each experiment, and thus are not amenable to use directly to test a large number of compounds for the ability to inhibit intracellular Hb S polymerization. For instance, approaches that use laser photolysis of carboxyhemoglobin in an anaerobic buffer to trigger sickling in individual cells (19), and then detect the sickling time by a microspectrophotometer suffer from low throughput due its cell-by-cell approach. In the methods described herein, for example, an assay is used that has the capability to measure accurately the sickling times for a large number of cells in a relatively short period of time and is readily scalable in order to facilitate the testing of a large number of compounds. In the methods of the instant invention, laser photolysis of carboxy-hemoglobin can be used as the method of triggering the polymerization process. Different from previous laser photolysis studies of sickle cells which only illuminated a single cell (19), the instant invention uses a defocused laser beam and a correspondingly larger illuminated field, and, thus, photolyzed simultaneously a large number of cells.

The invention also includes novel compounds identified by the above-described screening assays.

Optionally, such compounds are characterized in one or more appropriate animal models to determine the efficacy of the compound for the treatment of a neoplasia. Desirably, characterization in an animal model can also be used to determine the toxicity, side effects, or mechanism of action of treatment with such a compound. Furthermore, novel compounds identified in any of the above-described screening assays may be used for the treatment of a disease or disorder of peptide or protein polymerization, or abnormal peptide or protein polymerization, in a subject. Such compounds are useful alone or in combination with other conventional therapies known in the art.

### Methods of Treatment

As used herein, “treating” a disease or disorder of peptide or protein polymerization, or treating a disease of abnormal peptide or protein polymerization, refers to either slowing, stopping or reversing the progression of peptide or protein polymerization. In preferred embodiments, “treating” refers to treating sickle cell disease. “Treating” as used herein can mean reversing the progression to the point of eliminating the presence of peptide or protein polymerization, or in preferred embodiments the presence of sickled cells. As used herein, “treating” also means the reduction in the amount of polymerization of hemoglobin or the amelioration of symptoms associated with sickle cell disease. Alternatively, “treating” means arresting or otherwise ameliorating symptoms of peptide or protein polymerization, or abnormal peptide or protein polymerization, as defined herein.

Included in the invention are methods of identifying a subject having a disorder of peptide or protein polymerization. Accordingly, the methods can comprise aiming a laser at a cell population, and then obtaining obtaining images of the cell population, and analyzing the images to determine the kinetics of cell sickling, and thereby identifying a subject having a disorder of peptide or protein polymerization. The disease or disorder of peptide or protein polymerization can be a disorder of abnormal peptide or protein polymerization.

Also included in the invention are methods of treating a subject having a disease or disorder of peptide or protein polymerization. Accordingly, the methods can comprise treating the subject with an agent that inhibits peptide or protein polymerization, thereby treating the subject having a disorder of peptide or protein polymerization. The disease or disorder of peptide or protein polymerization can be a disorder of abnormal peptide or protein polymerization.

The invention also contemplates methods of treating a subject at risk of developing a disease or disorder characterized by peptide or protein polymerization. Accordingly, the method comprises administering to the subject an agent that inhibits peptide or protein polymerization, thereby treating the subject at risk of developing a disorder characterized by peptide or protein polymerization. The disease or disorder of peptide or protein polymerization can be a disorder of abnormal peptide or protein polymerization.

In any of the methods as described herein, the disorder of peptide or protein polymerization may be a disease or disorder effecting changes in cell size or shape. In certain embodiments, the disease or disorder may be a disease or disorder of abnormal peptide or protein polymerization. In other certain embodiments, the disorder of peptide or protein polymerization is selected from the group consisting of: sickle cell disease, Alzheimer's Disease, and Hereditary Spherocytosis. For example, the disorder of peptide or protein polymerization may be a sickle cell disease selected from, but not limited to Sickle SC Disease, Sickle Cell Anemia, and Sickle Cell Beta Disease. As used herein, the terms “sickle cell disease” means that the subject has at least one cell that contains Hemoglobin S (Hb S). As used herein, a “sickle cell” includes an erythrocyte from a subject with sickle cell disease in which polymerization of Hb S has occurred. As used herein, the term “sickling” is meant to include the process whereby polymerization of Hb S occurs. As used herein, the term “sickled” is meant to refer to an erythrocyte from a subject with sickle cell disease in which polymerization of HbS has occurred.

By way of example, a subject having a disease or disorder of peptide or protein polymerization, a subject at risk of developing a disease or disorder characterized by peptide or protein polymerization, or abnormal peptide or protein polymerization, as described herein can be treated as follows. An agent that inhibits peptide or protein aggregation can be administered to the patient, preferably in a biologically compatible solution or a pharmaceutically acceptable delivery vehicle, by ingestion, injection, inhalation, or any number of other methods. The dosages administered will vary from patient to patient; a “therapeutically effective dose” can be determined, for example, by monitoring the level of peptide or protein polymerization.

In the treatment of any disease or disorder of peptide or protein aggregation, as described herein, a therapeutically effective dosage regimen should be used. By “therapeutically effective”, one refers to a treatment regimen sufficient to restore the subject to the basal state, as defined herein, for instance at the cellular site of manifestation or to a disease or disorder of peptide or protein aggregation in an individual at risk thereof. Alternatively, a “therapeutically effective regimen” may be sufficient to arrest or otherwise ameliorate symptoms of peptide or protein aggregation. In the treatment of a disease or disorder of peptide or protein aggregation, or abnormal peptide or protein aggregation, an effective dosage regimen may require providing the medication over a period of time to achieve noticeable therapeutic effects.

This dosage may be repeated daily, weekly, monthly, yearly, or as considered appropriate by the treating physician.

Systemic administration of a therapeutic composition according to the invention may be performed by methods of whole-body drug delivery are well known in the art. These include, but are not limited to, intravenous drip or injection, subcutaneous, intramuscular, intraperitoneal, intracranial and spinal injection, ingestion via the oral route, inhalation, trans-epithelial diffusion (such as via a drug-impregnated, adhesive patch) or by the use of an implantable, time-release drug delivery device. Note that injection may be performed either by conventional means (i.e. using a hypodermic needle), or by hypospray (see Clarke and Woodland, 1975, Rheumatol. Rehabil., 14: 47-49).

Systemic administration is advantageous when a pharmaceutical composition must be delivered to a target that is widely-dispersed, inaccessible to direct contact or, while accessible to topical or other localized application, is resident in an environment (such as the digestive tract) wherein the native activity of the nucleic acid or other agent might be compromised, e.g. by digestive enzymes or extremes of pH.

It is contemplated that in certain cases global administration of a therapeutic composition to a subject may not be not needed in order to achieve a highly localized effect. Localized administration of a therapeutic composition according to the invention is preferably by injection or by means of a drip device, drug pump or drug-saturated solid matrix from which the composition can diffuse implanted at the target site. When a tissue that is the target of treatment according to the invention is on a surface of an organism, topical administration of a pharmaceutical composition is possible. For example, antibiotics are commonly applied directly to surface wounds as an alternative to oral or intravenous administration, which methods necessitate a much higher absolute dosage in order to counter the effect of systemic dilution, resulting both in possible side-effects in otherwise unaffected tissues and in increased cost.

Compositions comprising a therapeutic composition which are suitable for topical administration can take one of several physical forms, as summarized below:

(i) A liquid, such as a tincture or lotion, which may be applied by pouring, dropping or “painting” (i.e. spreading manually or with a brush or other applicator such as a spatula) or injection.

(ii) An ointment or cream, which may be spread either manually or with a brush or other applicator (e.g. a spatula), or may be extruded through a nozzle or other small opening from a container such as a collapsible tube.

(iii) A dry powder, which may be shaken or sifted onto the target tissue or, alternatively, applied as a nebulized spray.

(iv) A liquid-based aerosol, which may be dispensed from a container selected from the group that comprises pressure-driven spray bottles (such as are activated by squeezing), natural atomizers (or “pump-spray” bottles that work without a compressed propellant) or pressurized canisters.

(v) A carbowax or glycerin preparation, such as a suppository, which may be used for rectal or vaginal administration of a therapeutic composition.

Note that in some cases, the surface in question is internal; in such a case, topical application would comprise taking the therapeutic composition via an oral route, whether in liquid, gel or solid form.

A therapeutic composition of use in the invention can be given in a single- or multiple dose. A multiple dose schedule is one in which a primary course of administration can include 1-10 separate doses, followed by other doses given at subsequent time intervals required to maintain and or reinforce the level of the therapeutic agent. Such intervals are dependent on the continued need of the recipient for the therapeutic agent, and/or the half-life of a therapeutic agent. The efficacy of administration may be assayed by monitoring the reduction in the levels of a symptom indicative or associated with peptide or protein polymerization which it is designed to inhibit. The assays can be performed as described herein or according to methods known to one skilled in the art.

A therapeutically effective regimen may be sufficient to arrest or otherwise ameliorate symptoms of a disease. An effective dosage regimen requires providing the agent over a period of time to achieve noticeable therapeutic effects wherein symptoms are reduced to a clinically acceptable standard or ameliorated. For example, when the disease is associated with cell sickling, the claimed invention is successful when cell sickling time is increased by at least 10%. In certain embodiments, cell sickling time may be increased by 10%, 20%, 30%, 40%, 50%, 60%, 70%, 80%, 90%, 100,%, 200%, 300%, 400%, 500% or more. It is appreciated that the symptoms may be specific for the disease in question.

### Diseases

The methods of the invention as described herein have therapeutic and diagnostic applications to treat diseases. The methods as described herein can be used to identify a subject having a disorder of peptide or protein aggregation, or abnormal peptide or protein polymerization. The methods of the invention can also be use to treat a subject having a disease or disorder of peptide or protein polymerization, or to treat a subject at risk of developing a disease or disorder characterized by peptide or protein polymerization.

In general, the diseases or disorders that are amenable to the methods of the invention include disorders of peptide or protein polymerization. One of skill in the art will recognize that diseases of peptide or protein polymerization can be a disease or disorder effecting, or caused by, changes in cell size or shape. Thus, those of skill in the art will recognize that the methods may be used in other applications for which it is beneficial to treat diseases or disorders of peptide or protein polymerization.

For instance, in the allergic response, it has been shown that the H(4) receptor mediates eosinophil chemotaxis, cell shape change and upregulation of adhesion molecules. The effect of H(4) receptor antagonists in blocking eosinophil infiltration could be valuable for the treatment of allergic diseases. Further, the histamine-induced shape change and upregulation of adhesion molecules on eosinophils can serve as biomarkers for clinical studies of H(4) receptor antagonists (31). Other diseases or disorders that may be amenable to treatment according to the instant invention include for example, hereditary elliptocytosis or hereditary stomatocytosis. Hereditary elliptocytosis is usually inherited as an autosomal dominant trait. The defect is a structural abnormality of spectrin or a deficiency of the RBC membrane protein 4.1, without anemia and usually with no splenomegaly and only mild hemolysis. Most patients are asymptomatic. Hereditary stomatocytosis is an autosomal dominant disorder, in which a membrane abnormality leads to increased permeability to sodium. This causes water to move into the RBC decreasing the mean corpuscular hemoglobin concentration.

In certain preferred embodiments of the invention, the disease or disorder can be Sickle Cell Disease, Alzheimer's Disease, or Hereditary Spherocytosis. Other diseases or disorders that may be amenable to treatment according to the instant invention may include ageing and certain types of cancer.

Sickle Cell Disease

Sickle cell disease is one of the most prevalent hematologic genetic disorders in the world (3) (2) that occurs as a result of a single point mutation of Glu6 in Hb to Va16 in sickle hemoglobin (Hb S). Sickle cell disease is an inherited disorder that in patients results in periodic pain crises due to occlusion of capillaries by red blood cells (1). Sickle SC Disease, Sickle Cell Anemia, and Sickle Cell Beta Disease are all types of Sickle Cell Disease. At the molecular level, the disease is caused by a mutant form of hemoglobin (2,3), Hemoglobin S (Hb S), which, when deoxygenated, polymerizes with other Hb S molecules to form fibers. The formation of fibers inside red blood cells alters the shape (4) and mechanical properties of the cells (5,6) and can lead to vasocclusion. Vasoocclusion is the blockage of a capillary by a sickle cell, and results in ischemia and/or infarction. This process most frequently occurs in the spleen of patients with sickle cell disease, resulting in autosplenectomy.

Hb and HbS have almost identical positions for all amino acids, even in the A helix of the chains where the mutation occurs. The presence of the Va16 results in hydrophobic interaction between the mutation region of one Hb molecule and a region defined by Phe85 and Leu88 in the heme pocket of another Hb molecule. This interaction occurs only in the deoxygenated HbS (deoxyHbS), and induces polymerization of the deoxyHbS molecules into fibers. The formation of HbS polymers causes the normally flexible red blood cells to adopt rigid, sickle like shapes that block small capillaries and cause both local tissue damage and severe pain. The disease is also characterized by other symptoms, including hemolysis, which gives rise to anemia and jaundice, elevation of bilirubin level leading to high incidence of gall stones and impairment of hepatic excretory function. Other clinical features include leg ulceration, pneumonia, enlarged liver and spleen. Other studies on the gelatin of deoxyHbS and various Hb variants have also provided crucial information on other contact points on the Hb that are important in stabilizing the HbS fiber (34) (35). There are various therapeutic strategies to treat sickle cell disease (SCD), including (1) Pharmacological modulation of fetal hemoglobin (HbF): HbF has been shown to decrease HbS polymerization, and there are several agents that are known to induce HbF formation by possibly reactivating the genetic switch for HbF (36). Examples of such agents include 5-azacytidine, hydroxyurea and cytosine arabinoside (37). Unfortunately, there are serious toxic side effects associated with this therapy as a result of high doses and frequency of administration (38), (2) Bone marrow transplantation: Bone marrow transplant has also been used as a total gene replacement therapy for HbS in extreme cases (39) (40). This approach is very expensive and has its own inherent toxicities and risks (39), (3) Blood transfusion: This is one of the most common sickle cell disease therapies, however, repeated blood transfusions are known to be associated with the risks of infectious diseases, iron overload and allergic reactions (41), (4) Opioid analgesics: This therapy is necessary to deal with pain crisis, however, opioid therapy often results in addiction and/or seizures and/or depression, (41), (5) Erythrocyte membrane acting agents: Since the sickling process is partly dependent on intracellular concentration of sickle Hb, agents that induce cell swelling (42) or inhibit cell dehydration (43) could decrease the HbS concentration and help delay the polymerization process, and (6) Anti-gelling agent or HbS modifiers: These compounds interfere with the mechanism of polymerization by either binding directly to or near contact site(s) of the deoxyHb S to inhibit the polymerization process or act directly on HbS to shift the allosteric equilibrium to the more soluble high-affinity HbS.

In blood, Hb is in equilibrium between the tense (T) and the relaxed states. The Hb delivers oxygen via an allosteric mechanism, and the ability for the Hb to release or take oxygen can be regulated by allosteric effectors. When the allosteric equilibrium is shifted towards the relaxed state, a high-affinity Hb is obtained that more readily binds and holds oxygen while a shift toward the T state results in a low-affinity Hb that more easily releases oxygen. An increase in the naturally occurring allosteric effector, 2,3-DPG in red cells right shifts the oxygen equilibrium curve (OEC) as does an increase in temperature and decrease in pH (44). An increase in pH and lowering of the temperature and DPG levels left shifts the OEC. The degree of shift in the OEC is reported as an increase or decrease in P₅₀ (partial pressure of oxygen at 50% Hb saturation). Regulating the allosteric equilibrium to the relaxed conformation has been of been of interest in medicine. In particular, the identification of non-toxic compounds that efficiently bind to HbS and produce high-affinity HbS which does not polymerize have been clinically evaluated as antisickling agents to treat SCD. There is an ongoing need to identify such compounds to be used as antisickling agents to treat sickle cell anemia. See, for example, the use of vanillin (45), 12C79 (46), furfural (47), and substituted isothiocyanates (48).

The kinetics of Hb S polymerization are essential to understanding sickle cell disease on the molecular level (10). The kinetics of polymerization of in vitro solutions of Hb S have been studied extensively (11, 12), and Hb S has been shown to polymerize by a double nucleation mechanism (13). Deoxygenated Hb S molecules aggregate in solution and can form a stable nucleus by homogeneous nucleation (14) which is subsequently elongated into a fiber by additional Hb S molecules. New fibers can also result from heterogeneous nucleation wherein a nucleus forms on the surface of an extant fiber. In fact, because heterogeneous nucleation is a much more rapid process than homogeneous nucleation is, only a small fraction of all fibers in a suspension are formed by homogeneous nucleation. As a result of the double nucleation mechanism, Hb S polymerization is characterized by a delay time and a rapid growth phase. The delay time, which has the astonishing property that it varies inversely with up to the 40th power of concentration (11), is the period in which polymer is not detectable, and the rapid growth phase is the period in which soluble Hb S is converted to insoluble fibers. The delay time, or its cellular counterpart, the sickling time, has been shown to have therapeutic significance (15), as sickle cell disease is survivable if the sickling time of a cell is longer than the transit time of the cell through the microcirculation. Once the cell has passed into larger vessels, the risk of vasocclusion is abolished. Therefore one promising mechanism to treat sickle cell disease is to administer a drug that increases the sickling time of cells. In order to discover such a drug a sensitive kinetic assay of intracellular Hb S polymerization is required (9).

At the present moment, one of the only compounds approved by the United States Food and Drug Administration to treat sickle cell disease is hydroxyurea, a chemotherapy drug that provides a modest reduction in the mean number of pain crises per annum (7-9).

Hereditary Spherocytosis

Hereditary Spherocytosis, also known as congenital spherocytic anemia, is a disorder of the red blood cell membrane that leads to sphere-shaped red blood cells, and chronic hemolytic anemia, which is the premature breakdown of red blood cells. Hereditary Spherocytosis is caused by a defective gene. The defect results in an abnormal red cell membrane so that the affected cells have a smaller surface area for their volume than normal red blood cells. The cells are less resistant to stresses and rupture easily. The anemia varies in its severity. In severe cases the disorder may be detected in early childhood, or in mild cases it may go unnoticed until later in adult life.

This disorder is most common in people of northern European descent but has been found in all races. Jaundice and pallor (pale coloring) may be noted in infants, and the spleen is enlarged in most cases. After the spleen is removed, the life span of the red blood cell returns to normal. A family history of spherocytosis increases the risk for this disorder.

Symptoms of Hereditary Spherocytosis include jaundice, pallor, shortness of breath, fatigue, weakness, and irritability in children. Signs and test for the disease include physical examination to reveal enlarged spleen, an elevated reticulocyte count, blood smear to show spherocytes, anemia as determined by complete blood count shows anemia, osmotic fragility and incubated fragility test, Coombs' test wherein the direct is negative, Coombs' test wherein the indirect is negative, elevated bilirubin, elevated LDH.

Current treatment for the disease is splenectomy to cure the anemia of spherocytosis. Although the abnormal cell defect persists, the red cell life span returns to normal.

Because this is an inherited disorder and may not be preventable. Awareness of risk, such as a family history of the disorder, may allow early diagnosis and treatment. Information, as described herein, is available publicly on the world wide web at nlm.nih.gov/medlineplus/ency/article/000530.

Alzheimer's Disease

Alzheimer's disease (AD) is a progressive, neurodegenerative disease characterized in the brain by abnormal clumps (amyloid plaques) and tangled bundles of fibers (neurofibrillary tangles) composed of misplaced and aggregated proteins. Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function. The early symptoms of AD, which include forgetfulness and loss of concentration, are often missed because they resemble natural signs of aging.

Currently, there is no cure for AD and no way to slow the progression of the disease. For some people in the early or middle stages of AD, medication such as tacrine (Cognex) may alleviate some cognitive symptoms. Donepezil, rivastigmine, and galantamine may keep some symptoms from becoming worse for a limited time. A fifth drug, memantine, was recently approved for use in the United States. Combining memantine with other AD drugs may be more effective than any single therapy. One controlled clinical trial found that patients receiving donepezil plus memantine had better cognition and other functions than patients receiving donepezil alone. Also, other medications may help control behavioral symptoms such as sleeplessness, agitation, wandering, anxiety, and depression.

An early, accurate diagnosis of AD helps patients and their families plan for the future. It gives them time to discuss care while the patient can still take part in making decisions. Early diagnosis will also offer the best chance to treat the symptoms of the disease. Thus, the methods of the instant invention will can, in one embodiment, provide a method of determining a patient at risk for AD by determining, for instant misplaced and aggregated or polymerized proteins at an early stage of progression of the disease. Likewise, the methods of the invention can provide methods of identifying novel agents to treat AD by inhibiting said misplaced and aggregated or polymerized proteins underlying the pathology of the disease. Information about Alzheimer's Disease is publicly available on the world wide web at ninds.nih.gov/disorders/alzheimersdisease/alzheimersdisease.htm.

### Compositions

The invention provides for compositions comprising a compound according to the invention, and a physiologically compatible carrier. In certain embodiments, the invention provides a composition comprising an agent that inhibits peptide or protein polymerization, and a pharmaceutically acceptable carrier. In other embodiments, the invention provides a composition comprising an agent that inhibits cell sickling, and a pharmaceutically acceptable carrier.

As used herein, “pharmaceutically acceptable carrier” refers to a physiologically acceptable diluent such as water, phosphate buffered saline, or saline, and further may include an adjuvant. Adjuvants such as incomplete Freund's adjuvant, aluminum phosphate, aluminum hydroxide, or alum are materials well known in the art. In addition to the active ingredients, pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the subject.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic and succinic. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a Ph range of 4.5 to 5.5 that is combined with buffer prior to use.

After pharmaceutical compositions comprising a compound of the invention formulated in an acceptable carrier have been prepared, they can be placed in an appropriate container and labeled for treatment of an indicated condition with information including amount, frequency and method of administration.

The, pharmaceutical compositions comprising a compound of the invention as described herein can further comprise one or more therapeutic agents. Exemplary agents that can be combined with the compositions of the invention as described herein include therapeutic agents known in the art for the treatment of diseases or disorders characterized by peptide or protein polymerization, or abnormal peptide or protein polymerization, for example sickle cell disease, including Sickle SC Disease, Sickle Cell Anemia, and Sickle Cell Beta Disease, Alzheimer's Disease or Hereditary Spherocytosis.

For example, 5-hydroxymethyl-2-fufural, a five carbon ring aromatic aldehyde that exists naturally in coffee, honey, dried fruits fruit juices and flavoring agents, has been described to inhibit sickling of sickle cells. U.S. Pat. No. 7,119,208, incorporated by reference herein, describes anti-sickling agents based on naturally occurring 5-hydroxymethyl-2-furfuraldehyde (5HMF or AMS-13), 5-Ethyl-2-furfuraldehyde (5EF), 5-Methyl-2-furfuraldehyde (5MF) and 2-furfuraldehyde (FUF), including analogues and derivatives of these compounds.

Additionally, antiviral compounds to inhibit cell sickling may be used in combination with the compositions of the invention. For example, as described in U.S. Pat. No. 6,946,457, the antiviral compounds acyclovir or valacyclovir may be used. The antiviral agent may be a purine analog, which includes any compound which comprises a purine group. The purine analog may be a guanosine analog, which includes any compound which comprises a guanosine group. Acyclovir and valacyclovir are examples of guanosine analogs.

Other agents that may be useful in combination with the compositions of the instant invention are described in U.S. Pat. No. 6,184,228, incorporated by reference herein in its entirety, and include HbS ligands, which may be an isoquinolium derivatives, exhibiting inhibition of HbS polymerization and displaying anti-sickling activity of red blood cells. U.S. Pat. No. 4,904,678, incorporated herein by reference in its entirety, describes the use of specified amounts of thiocyanate salts of either alkali metals or ammonium, or these salts together with pyridoxine hydrochloride (vitamin B₆).

### Kits

The present compositions may be assembled into kits. In certain embodiments, kits according to the invention may comprise an agent that inhibits peptide or protein polymerization according to the methods described herein, and instructions for use. In other embodiments, kits according to the invention may comprise an agent that inhibits cell sickling according to the methods described herein, and instructions for use.

Kits or pharmaceutical systems according to this aspect of the invention comprise a carrier means, such as a box, carton, tube or the like, having in close confinement therein one or more container means, such as vials, tubes, ampules, bottles and the like.

### EXAMPLES

The invention will now be further illustrated with reference to the following methods and examples. It will be appreciated that what follows is by way of example only and that modifications to detail may be made while still falling within the scope of the invention.

### Example 1

### A Method to Measure Distributions of Sickling Times for a Population of Red Blood Cells In An Accurate and Sensitive Manner

The present work describes methods that have the capability to measure accurately the sickling times for a large number of cells in a relatively short period of time and is readily scalable in order to facilitate the testing of a large number of compounds to identify an inhibitor of cell sickling. In the methods of the instant invention, the are a number of independent dimensions of control that can be identified as follows: (1) the number of cells, (2) the methods of triggering polymerization that include laser, chemical and slow deoxygenation, (3) treatment with a drug, (4) acquisition of data, (5) analysis of data and (6) automation, for example by high throughput means.

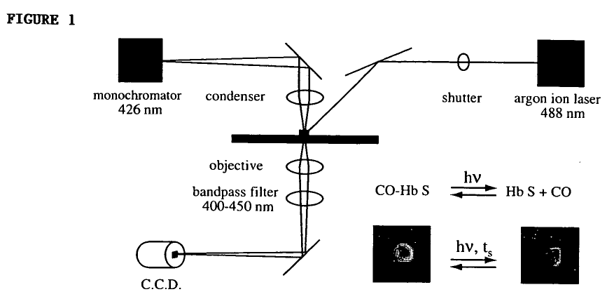

An exemplary method according to the invention is shown schematically in FIG. 1. Here, laser photolysis of carboxy-hemoglobin (Hb)was selected as the method of triggering the polymerization process. However, unlike in previous laser photolysis studies of sickle cells which only illuminated a single cell (19), the present study utilized a defocused laser beam and corresponding larger illuminated field, and, thus, photolyzed simultaneously a large number of cells. Data were acquired in the form of a series of bright field images in an optical microscope both prior to and after the opening of a shutter in the beam path (see also description in Methods section below). Briefly, an argon ion laser tuned to emit at 488 nm and aimed at a 45° angle relative to the sample was used to photodissociate the hemoglobin in a large field of red blood cells to the deoxygenated state, and thus serve as an optical trigger to initiate polymerization of hemoglobin-S (Hb-S) digital images were acquired on a CCD and analyzed subsequently using the algorithm described herein to obtain sickling time distributions, the key kinetic parameter of intracellular Hb S polymerization, and thus sickle cell disease.

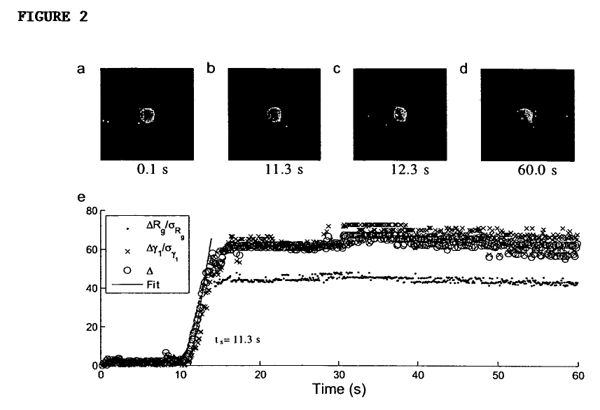

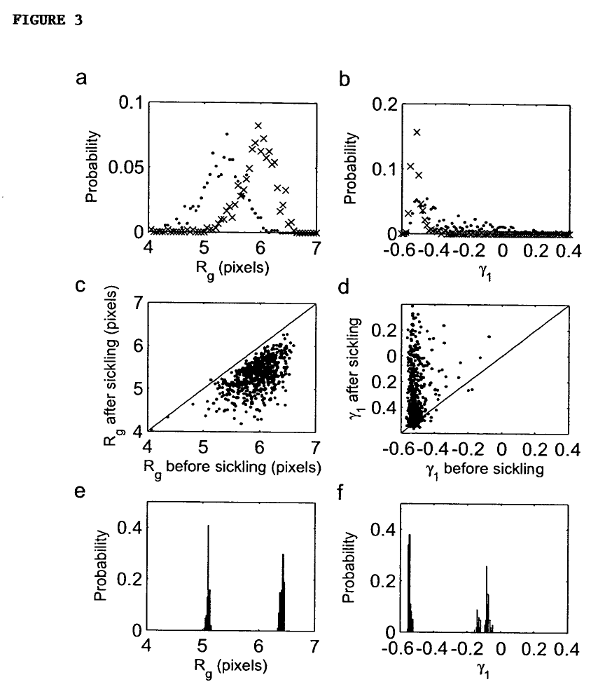

In order to measure quantitatively the kinetics of sickling for a given sample, and thus produce distributions of sickling times, an algorithm was developed to analyze the acquired digital images and measure accurately the sickling times of each cell in the field of view (FIG. 2a-e). Red blood cells in the images were identified using a Sobel mask (20), and objects that were either on the border of the image, or did not have an area consistent with that of a single cell, were excluded from the analysis. A particular cell was matched in the series of images by sequentially matching an object in a particular image with the nearest object in the following image, and then repeating this process across all images.

Two parameters measuring the size and shape of the identified object, the radius of gyration of a cell and the skewness of the radial distribution of pixels in a cell (FIG. 3a-f), respectively, were measured for each cell in the series of images and thus as a function of time. For each cell, fluctuations in the two parameters were measured prior to opening the shutter, and baseline mean values and standard deviations were calculated (see Methods). Upon opening the shutter, carbon monoxide was photodissociated from hemoglobin, deoxy-hemoglobin S began to polymerize, and cells began to distort due to the presence of fibers (not shown). Concurrently, significant changes in the size and shape of cells, relative to the respective baseline mean values and standard deviations prior to opening the shutter, were observed. The point where the rate of change in these parameters was maximal was identified, and the sickling time was determined by extrapolating the slope from the measured relative change in the parameter values back to the point of intersection with the time axis. A small fraction of cells did not have a significant degree of distortion during the observation period, and were thus identified as unsickled by the algorithm.

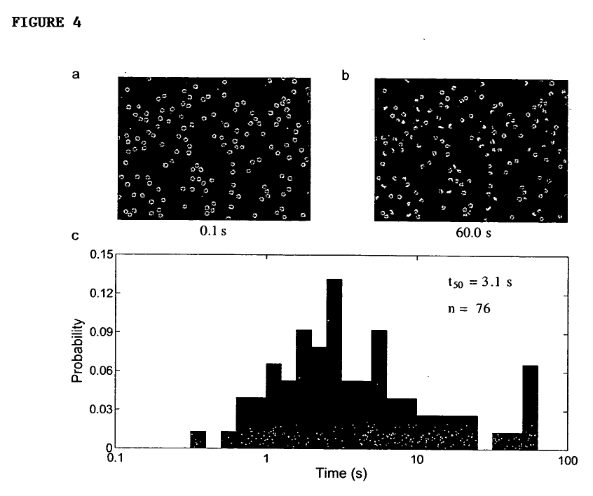

Given the extraordinary sensitivity of nucleation rates on Hb S concentration (21) and the distribution of hemoglobin concentration known to exist in red blood cells (22), it is necessary to have distributions of sickling times for a population of cells in order to be able to properly quantify intracellular Hb S polymerization. A distribution of sickling times (FIG. 4a-c) was constructed readily by compiling the sickling times of all detectable red blood cells in the field of view. Using this approach with a standard 20× objective lens and 8 bit VGA CCD , kinetic data on the polymerization of approximately 50-100 cells could be acquired at one time (data not shown). The assay and associated analysis algorithm are directly scalable to multi-well plate format, and therefore have the potential to be used as the basis for a large scale screen for inhibitors of sickle cell disease.

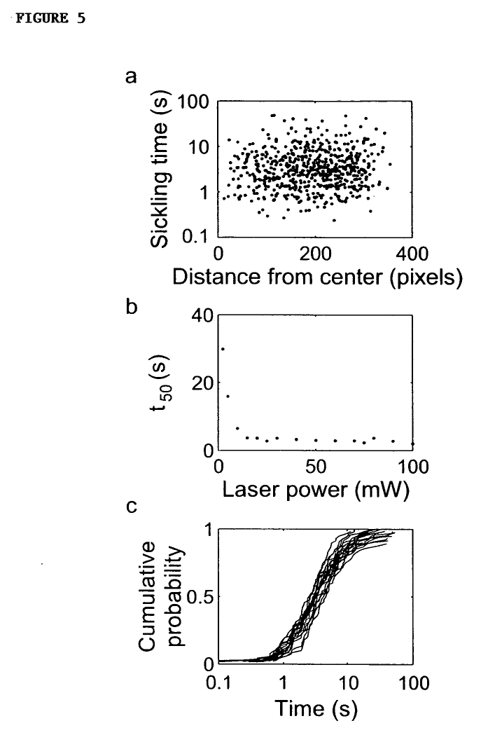

In order to test the reliability of the assay, a series of control experiments were performed, as shown in FIG. 5a-c. First, the correlation coefficient of the distance from the center of the image for each cell with its measured sickling time was calculated (FIG. 5a). The two variables were found to be uncorrelated. Second, the fraction of intracellular deoxy-Hb S was shown to be controlled by the laser intensity, and a very moderate laser intensity, along with the use of an anaerobic buffer, was capable of fully deoxygenating the hemoglobin in the cell (FIG. 5b). Third, 20 independent laser photolysis experiments demonstrated that the sickling time distributions were nearly identical in all cases (FIG. 5c).

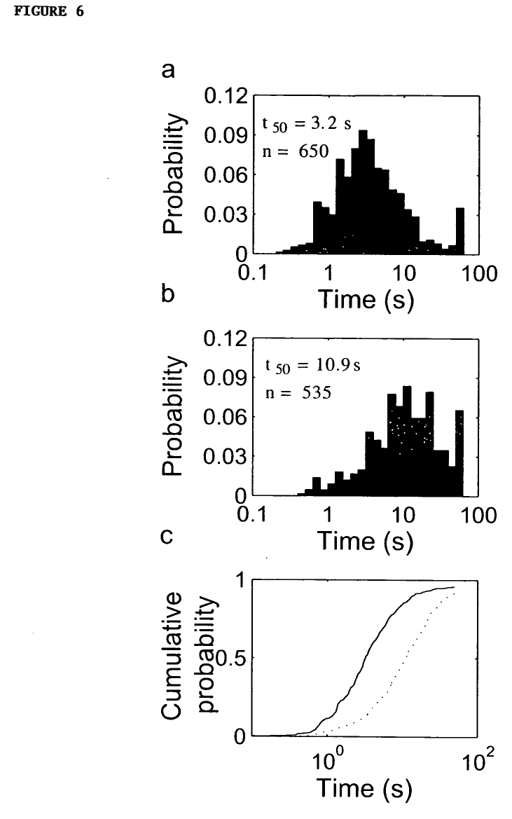

These data show an important feature of the assay described herein, which is its high sensitivity to detect changes in the sickling time distribution of red blood cells, a requisite characteristic for identifying an inhibitor of Hb S polymerization in a large scale screen. FIG. 6, shows the sensitive detection of differences in sickling time distributions. Two dilutions of red blood cells heterozygous for Hb S were prepared in deoxygenated phosphate buffers, one with an osmolality of 365 mOsm and a second with an osmolality of 347 mOsm. The second sample was prepared by decreasing the osmolality of the buffer by 5% in order to increase the volume of the cell, and, thus, lower the hemoglobin concentration and increase sickling times. The last bins in each probability distribution corresponds to the number of unsickled cells at the end of the experiment. Comparison of the cumulative probability distributions (FIG. 6a-c) shows the exquisite sensitivity of intracelllular polymerization of Hb S on Hb S concentrations (solid curve, 365 mOsm; dotted curve, 347 mOsm) and the ability of the present method to detect readily the resulting changes in intracellular polymerization kinetics. The difference in the sickling time distributions for the two samples is made very evident (FIG. 6c) by this sensitive assay. As a result of the unusual kinetics of Hb S polymerization, the small perturbation in solution conditions was sufficient to produce more than a three-fold increase in the median sickling time.

Taken together, this data shows an assay that has the capability to measure accurately and sensitively the sickling times for a large number of cells in a relatively short period of time.

### Example 2

### A Method to Quantify the Degree of Cellular Distortion and the Duration of the Growth Phase

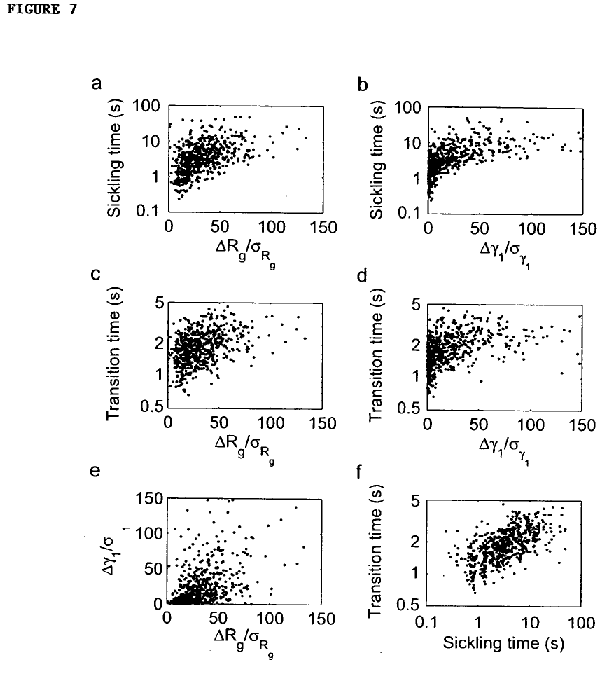

In addition to measuring distributions of sickling times for a population of red blood cells in an accurate and sensitive manner, the application of the method described in this paper also has the capabilities to quantify the degree of cellular distortion and the duration of the growth phase (FIG. 7), in ways that are inaccessible by other techniques (23, 24). The results show that those cells with the lowest sickling times undergo the least distortion and grow the fastest. This suggests that in vivo, where the cells that sickle the fastest are responsible for the onset of vasocclusion, cellular distortion per se is likely to be of secondary importance in causing sickle cell disease to that of the change in mechanical properties of the cell after the formation of fibers. Therefore, the development of this new method to measure sickling times of red blood cells has the possibility to contribute to the underlying scientific knowledge of the polymerization of Hb S as well as to the search for a more effective treatment of for sickle cell disease.

### Methods

### Sample Preparation

Human red blood cell samples were obtained and diluted in a phosphate buffer. An isotonic phosphate buffer was prepared as described in the literature (19), and the osmolality was increased to 365 mOsm, as determined by freezing point depression osmometry, by the addition of NaCl. The diluted red blood cells were stored in a glass vial with septum, and a humidified gas mixture of 1% CO and 99% N2 was flown into the vial. After equilibration of the buffer with the gas mixture, the diluted red blood cells were delivered anaerobically to a flow cell with glass coverslips and maintained at 37 C.

### Image Acquisition

Bright field images for all Figures were acquired on a Nikon TMD microscope using a monochromatic light source at 426 nm, the point where carboxy-and deoxy-hemoglobin have identical absorption values. An argon ion laser tuned to emit at 488 nm and laser power of 75 mW (except for FIG. 6B where the laser power was varied) with computer controlled shutter was used to photodissociate carboxy-hemoglobin to deoxy-hemoglobin at a 45° angle relative to the sample. The laser spot size was set to 1 mm². A bandpass filter (400-450 nm) was placed between the objective lens and CCD. Images were digitized on a CCD; an 8 bit VGA (640×480 pixels) CCD was used to acquire images with the 20× objective lens, and a 10 bit UXGA (1600×1200 pixels) CCD was used to acquire images with the 10× objective lens.

### Analysis of Digital Images

Digital images were analyzed by first stretching the grayscale to the full range (8 or 10 bits) and then using a Sobel mask to detect objects. Objects on an edge of an image and objects corresponding to the area of more than one cell were removed from the analysis. For each identified object the center of mass, radius of gyration (Rg) and skewness of the radial distribution (γ₁) were calculated. The radius of gyration was calculated as

$${R}_{g} = \sqrt{\frac{{\sum }_{j} {i}_{j} {r}_{j}^{2}}{{\sum }_{j} {i}_{j}}} ,$$

where iⱼ is the grayscale intensity of pixel j and rⱼ is the distance of pixel j from the identified object's center of mass. The skewness (30) was calculated as

$${γ}_{1} = \frac{{μ}_{r}^{( 3 )}}{{σ}_{r}^{3}}$$

where μᵣ⁽³⁾ is the third moment of the radial distribution of pixels and σᵣ³ is the standard deviation of the radial distribution of pixels. For a disk, the skewness can be determined analytically as

$${γ}_{1} = \frac{- {18}^{\frac{3}{2}}}{135} \approx - 0.57 .$$

Across images in a photolysis experiment, an object in one frame was matched to the object in the next frame with the nearest center of mass. Only objects that were uniquely identified across all frames of the sequence were analyzed.

Prior to the opening of the shutter on the laser, mean values and standard deviations were calculated for Rg and γ₁ for each cell in the field of view, allowing for Gaussian random variables,

$$\frac{Δ {R}_{g}}{{σ}_{{R}_{g}}} and \frac{Δ {γ}_{1}}{{σ}_{{γ}_{1}}}$$

with mean value of 0 and standard deviation of 1 to be created. In addition, Δ was calculated as

$$Δ = \frac{1}{\sqrt{2 - 2 ρ}} ( \frac{{Δγ}_{1}}{{σ}_{{R}_{g}}} - \frac{Δ {R}_{g}}{{σ}_{{R}_{g}}} )$$

where ρ is the empirically determined correlation coefficient of the two random variables. Δ was then smoothed by averaging over a three frame window. The smoothed Δ was then numerically differentiated. The maximal slope was then extrapolated back to the time axis from its position on Δ, and the resulting value was stored as the sickling time of the object. Cells whose maximal value of Δ≦5 were classified as unsickled during the observation period. The transition time was calculated as the maximum of Δ divided by the maximal slope.

Variations, modifications, and other implementations of what is described herein will occur to those of ordinary skill in the art without departing from the spirit and scope of the invention and claims herein. All patents, patent publications, international applications, and references are incorporated by reference herein in their entireties.

### REFERENCES

The following documents, also incorporated by reference, are indicated in the examples and discussion above by a number in parentheses.
1. Bunn, H. F. Pathogenesis and treatment of sickle cell disease. N Engl J Med 337, 762-769 (1997).
2. Pauling, L., Itano, H., Singer, S. J. & Wells, I. C. Sickle cell anemia, a molecular disease. Science 110, 543-548 (1949).
3. Ingram, V. M. Gene mutations in human haemoglobin: the chemical difference between normal and sickle cell haemoglobin. Nature 180, 326-328 (1957).
4. Sherman, I. J. The sickling phenomenon, with special reference to the differentiation of sickle cell anemia from sickle cell trait. Bull. Johns Hopkins Hospital 67, 309-324 (1940).
5. Clark, M. R., Mohandas, N. & Shohet, S. B. Deformability of oxygenated irreversibly sickled cells. J Clin Invest 65, 189-196 (1980).
6. Lucas, G. S., Caldwell, N. M. & Stuart, J. Fluctuating deformability of oxygenated sickle erythrocytes in the asymptomatic state and in painful crisis. Br J Haematol 59, 363-368 (1985).
7. Charache, S. et al. Effect of hydroxyurea on the frequency of painful crises in sickle cell anemia investigators of the multicenter study of hydroxyurea in sickle cell anemia. N Engl J Med 332, 1317-1322 (1995).
8. Bridges, K. R. et al. A multiparameter analysis of sickle erythrocytes in patients undergoing hydroxyurea therapy. Blood 88, 4701-4710 (1996).
9. Eaton, W. A. & Hofrichter, J. The biophysics of sickle cell hydroxyurea therapy. Science 268, 1142-1143 (1995).

10. Eaton, W. A. & Hofrichter, J. Sickle cell hemoglobin polymerization. Adv. Protein. Chem. 40, 63-279 (1990).
11. Hofrichter, J., Ross, P. D. & Eaton, W. A. Kinetics and mechanism of deoxyhemoglobin s gelation: a new approach to understanding sickle cell disease. Proc Natl Acad Sci U S A 71, 4864-4868 (1974).
12. Ferrone, F. A., Hofrichter, J. & Eaton, W. A. Kinetics of sickle hemoglobin polymerization. i. studies using temperature-jump and laser photolysis techniques. J Mol Biol 183, 591-610 (1985).
13. Ferrone, F. A., Hofrichter, J. & Eaton, W. A. Kinetics of sickle hemoglobin polymerization. ii. a double nucleation mechanism. J Mol Biol 183, 611-631 (1985).
14. Galkin, O. & Vekilov, P. G. Mechanisms of homogeneous nucleation of polymers of sickle cell anemia hemoglobin in deoxy state. J Mol Biol 336, 43-59 (2004).
15. Mozzarelli, A., Hofrichter, J. & Eaton, W. A. Delay time of hemoglobin s polymerization prevents most cells from sickling in vivo. Science 237, 500-506 (1987).
16. Horiuchi, K., Ohata, J., Hirano, Y. & Asakura, T. Morphologic studies of sickle erythrocytes by image analysis. J Lab Clin Med 115, 613-620 (1990).
17. Horiuchi, K. et al. Image analysis studies of the degree of irreversible deformation of sickle cells in relation to cell density and Hb F level. Br J Haematol 85, 356-364 (1993).
18. Zarkowsky, H. S. & Hochmuth, R. M. Sickling times of individual erythrocytes at zero po2. J Clin Invest 56, 1023-1034 (1975).
19. Coletta, M., Hofrichter, J., Ferrone, F. A. & Eaton, W. A. Kinetics of sickle haemoglobin polymerization in single red cells. Nature 300, 194-197 (1982).
20. Gonzalez, R. C. & Woods, R. E. Digital Image Processing (Prentice Hall, Upper Saddle River, N.J., 2002), 2 edn.
21. Corbett, J. D., Mickols, W. E. & Maestre, M. F. Effect of hemoglobin concentration on nucleation and polymer formation in sickle red blood cells. J Biol Chem 270, 2708-2715 (1995).
22. Lew, V. L., Raftos, J. E., Sorette, M., Bookchin, R. M. & Mohandas, N. Generation of normal human red cell volume, hemoglobin content, and membrane area distributions by “birth” or regulation? Blood 86, 334-341 (1995).
23. Asakura, T. & Mayberry, J. Relationship between morphologic characteristics of sickle cells and method of deoxygenation. J Lab Clin Med 104, 987-994 (1984).
24. Asakura, T., Asakura, K., Obata, K., Mattiello, J. & Ballas, S. K. Blood samples collected under venous oxygen pressure from patients with sickle cell disease contain a significant number of a new type of reversibly sickled cells: constancy of the percentage of sickled cells in individual patients during steady state. Am J Hematol 80, 249-256 (2005).
25. Eaton, W. A. Linus pauling and sickle cell disease. Biophys Chem 100, 109-116 (2003).
26. Dykes, G., Crepeau, R. H. & Edelstein, S. J. Three-dimensional reconstruction of the fibres of sickle cell haemoglobin. Nature 272, 506-510 (1978).
27. DeBellis, R. H., Chen, B.-X. & Erlanger, B. F. Inhibition of sickling in vitro by three purine-based antiviral agents: an approach to the treatment of sickle cell disease. Blood Cells Mol Dis 31, 286-290 (2003).
28. Zhang, C. et al. Anti-sickling effect of MX-1520, a prodrug of vanillin: an in vivo study using rodents. Br J Haematol 125, 788-795 (2004).
29. Abdulmalik, O. et al. 5-hydroxymethyl-2-furfural modifies intracellular sickle haemoglobin and inhibits sickling of red blood cells. Br J Haematol 128, 552-561 (2005).
30. Larsen, R. J. & Marx, M. L. An Introduction to Mathematical Statistics and Its Applications (Prentice Hall, Upper Saddle River, N.J., 2005), 4 edn.
31. Ling P. Ngo K, Nguyen S, Thurmond R L, Edwards J P, Karlsson L, Fung-Leung W P. Histamine H4 receptor mediates eosinophil chemotaxis with cell shape change and adhesion molecule upregulation. Br J Pharmacol. May 2004; 142(1):161-71.
32. Ferster, A., C. Vermylen, G. Cornu, M. Buyse, F. Corazza, C. Devalck, P. Fondu, M. Toppet, E. Sariban (1996), “Hydroxyurea For Treatment Of Severe Sickle Cell Anemia: A Pediatric Clinical Trial”, Blood 88: 1960-1964.
33. Ha K S, Yeo E J, Exton J H. Lysophosphatidic acid activation of phosphatidylcholine-hydrolysing phospholipase D and actin polymerization by a pertussis toxin-sensitive mechanism. Biochem J. Oct. 1, 1994; 303(Pt 1): 55-59
34. Adachi, K., and Asakura, T. (1980) Polymerization of deoxyhemoglobin C Harlem (.beta.6Glu-Val, .beta.73Asp-Asn), J. Mol. Biol., 144, 467.
35. Bunn, H. F., and Forget, G. B. (1986) Hemoglobin: Molecular, Genetic and Clinical Aspects, p 462, W. B. Saunders company.
36. Olivieri, N. F., Weatherall, D. J. (1998). The therapeutic reactivation of fetal haemoglobin. Hum. Mol. Genet. 7, 1655.
37. Mehanna, A. S. Sickle cell anemia and antisickling agents then and now. (2001) Curr. Med. Chem. 8, 79.
38. Edelstein, S. J. Chapter 25, Sickle Cell Anemia In Ann. Reports in Med. Chem. Bailey, D. M., Ed.; Academic Press, Inc. 20, 247, (1985).
39. Hillery, C. A. (1998) Potential therapeutic approaches for the treatment of vaso-occlusion in sickle cell disease. Curr. Opin. Hematol., 5, 151.
40. Johnson, F. L. (1985) Bone marrow transplantation in the treatment of sickle cell anemia. Am. J. Pediatr. Hematol. Oncol. 7, 254.
41. Ballas, S. K. (1999) Complications of sickle cell anemia in adults: guidelines for effective management. Clev. Clin. J. Med., 66, 48.
42. Asakura, T., Ohnishi, S. T., Adachi, K., Ozgul, M., Hashimoto, M., Singer, M., Russell, M. O., Schwartz, E. (1980) Effect of cetiedil on erythrocyte sickling: new type of antisickling agent that may affect erythrocyte membranes. Proc. Natl. Acad. Sci., USA, 77, 2955.
43. Orringer, E. P., Berkwitz, L. R. (1986) In Approaches to the therapy of sickle cell anemia, Beuzard, Y.; Charache, S., Galacteros, F., Eds.; Les Edition Inserum: Paris, 141, 301.
44. Reeves, R. B. (1980) The effect of temperature on the oxygen equilibrium curve of human blood, Resir. Physiol., 42, 317.
45. Abraham, D. J., Mehanna, A. S., Wireko, F. C., Whitney, J., Thomas, R. P., and Orringer, E. P. (1991). Vanillin, a potential agent for the treatment of sickle cell anemia, Blood 77, 1334.
46. Fitzharris, P., McLean, A. E., Sparks, R. G., Weatherley, B. C., White, R. D., and Wootton, R. (1985) The effects in volunteers of BW12C, a compound designed to left-shift the blood-oxygen saturation curve, Br. J. Clin. Pharmacol. 19, 471.
47. Zuagg, R. H, Walder, J. A, and Klotz, I. M. (1977) Schiff Base Adducts of Hemoglobin Modifications that inhibit erythrocyte sickling, J. Biol. Chem. 252, 8542.
48. Park, S., Hayes, B. L., Marankan, F., Mulhearn, D. C., Wanna, L., Mesecar, A. D., Santarsiero, B. D., Johnson, M. E., and Venton, D. L. (2003) Regioselective covalent modification of hemoglobin in search of antisickling agents, J. Med. Chem. 46, 936.

What is claimed is:

1. A method for detecting cell sickling or peptide or protein polymerization, the method comprising:
aiming a light source at a cell population;
obtaining images of the cell population; and
analyzing the images to determine the kinetics of sickling of the cells;
thereby detecting cell sickling, peptide or protein polymerization in the cell population.

2. A method for identifying an inhibitor of cell sickling, peptide or protein polymerization, the method comprising:
aiming a light source at a cell population;
contacting the cell population with a candidate agent; and
determining the kinetics of cell sickling;
thereby identifying an inhibitor of cell sickling, peptide or protein polymerization.

3. The method of claim 1 or claim 2, wherein the light source is a laser beam.

4. The method of claim 1 or claim 2, wherein the step of aiming the light source at a cell population activates polymerization of deoxyhemoglobin-S.

5. The method of claim 1, wherein the kinetics of cell sickling is compared to the kinetics of cell sickling in a cell population that was not treated with agent.

6. The method of claim 1, wherein the analyzing comprises measuring the size and shape of cells in the population.

7. The method of claim 6, wherein the size is measured by the radius of gyration of the cell.

8. The method of claim 6, wherein the shape is measured by the skewness of the radial distribution of pixels in a cell.

9. A method for identifying a subject having a disorder of peptide or protein polymerization, the method comprising:
inducing polymerization in a cell population;
obtaining images of the cell population; and
analyzing the images to determine the kinetics of cell sickling;
thereby identifying a subject having a disorder of peptide or protein polymerization.

10. The method of claim 9, wherein polymerization is induced by aiming a light source at the cell population.

11. A method for treating or preventing a disease or disorder of peptide or protein polymerization in a subject, the method comprising:
administering to the subject an effective amount of agent that inhibits peptide or protein polymerization; and
thereby treating or preventing a disease or disorder of peptide or protein polymerization.

12. The method of claim 11, wherein the disorder of peptide or protein polymerization is selected from the group consisting of: sickle cell disease, Alzheimer's disease, Parkinson's disease, Huntington's disease, diseases associated with a prion protein, Hereditary Spherocytosis, and the class of diseases known as the hereditary amyloidoses.

13. A method for analyzing the characteristics of a cell population for cell sickling or cell polymerization, the method comprising:
obtaining images of the cell population; and
analyzing the images to determine the kinetics of sickling of the cells;
thereby analyzing the characteristics of a cell population for cell sickling or cell polymerization.

14. The method of claim 13, further comprising the step of obtaining a sample cell population.

15. The method of claim 14, wherein the cell population is human red blood cells.

16. A composition comprising an agent that inhibits cell sickling, peptide or protein polymerization, and a pharmaceutically acceptable carrier.

17. A kit comprising an agent that inhibits cell sickling, peptide or protein polymerization, and instructions for use.
